(19) United States
(12) Patent Application Publication
(10) Pub. No.: US 2024/0268705 A1
(43) Pub. Date: Aug. 15, 2024

(54) ORAL FUNCTION EVALUATION METHOD, RECORDING MEDIUM, ORAL FUNCTION EVALUATION DEVICE, AND ORAL FUNCTION EVALUATION SYSTEM

(71) Applicant: Panasonic Intellectual Property Management Co., Ltd., Osaka (JP)

(72) Inventors: Masashi ISHIMARU, Osaka (JP); Junko NAKAJIMA, Osaka (JP); Jakusei KIYOSAKI, Hyogo (JP); Yoshihiro MATSUMURA, Osaka (JP); Yaming ZHANG, Hyogo (JP); Takeshi KIKUTANI, Tokyo (JP); Makoto KARIYASU, Miyazaki (JP)

(21) Appl. No.: 18/563,251

(22) PCT Filed: Apr. 12, 2022
(86) PCT No.: PCT/JP2022/017643

§ 371 Date: Nov. 21, 2023

(30) Foreign Application Priority Data
May 31, 2021 (JP) 2021-091766

(51) Int. Cl. A61B 5/11; A61B 5/00; G16H 20/00

(52) U.S. Cl. CPC A61B 5/11; A61B 5/4803; G16H 20/00; A61B 2503/08

(57) ABSTRACT

An oral function evaluation method includes: obtaining voice data obtained by collecting a voice of an evaluatee uttering a syllable or a fixed phrase that includes (i) two or more morae including a change in a first formant frequency or a change in a second formant frequency or (ii) at least one of a flap, a plosive, a voiceless sound, a double consonant, or a fricative; extracting a prosody feature from the voice data obtained; calculating an estimated value of an oral function of the evaluatee, based on the prosody feature extracted and an oral function estimating equation calculated based on a plurality of training data items; and evaluating an oral function deterioration state of the evaluatee by assessing the estimated value using an oral function evaluation indicator.

### CROSS-REFERENCE OF RELATED APPLICATIONS

This application is the U.S. National Phase under 35 U.S.C. § 371 of International Patent Application No. PCT/JP2022/017643, filed on Apr. 12, 2022, which in turn claims the benefit of Japanese Patent Application No. 2021-091766, filed on May 31, 2021, the entire disclosures of which Applications are incorporated by reference herein.

### TECHNICAL FIELD

The present invention relates to an oral function evaluation method, a recording medium, an oral function evaluation device, and an oral function evaluation system which can evaluate an oral function of an evaluatee.

### BACKGROUND ART

A method to evaluate the eating and swallowing function of an evaluatee by obtaining a pharynx movement feature as an eating and swallowing function evaluation indicator (marker) from an appliance which is put on the neck of the evaluatee to evaluate the eating and swallowing function is disclosed (e.g., see Patent Literature (PTL) 1).

### CITATION LIST

### Patent Literature

[PTL 1] Japanese Unexamined Patent Application Publication No. 2017-23676

### SUMMARY OF INVENTION

### Technical Problem

However, the method disclosed in PTL 1 requires an evaluatee to put on the appliance to evaluate an oral function such as the eating and swallowing function. This may cause discomfort to the evaluatee and impose a burden on the evaluatee. An oral function can be evaluated also by visual inspection, interview, palpation, or the like by a specialist such as a dentist, a dental hygienist, a speech pathologist, or a physician. However, oral function deterioration of an elderly person may be overlooked, being regarded as a natural symptom of an elderly person, although the elderly person chokes all the time or spills food because of an influence of aging. Overlooking oral function deterioration brings about, for example, undernutrition resulting from a decrease in an amount of food intake, and the undernutrition brings about a decrease in immune strength. In addition, oral function deterioration tends to cause aspiration, and as a result, the aspiration and the decrease in immune strength bring about a vicious circle that leads to a risk that reaches aspiration pneumonia.

In view of the above, the present invention has an object to provide an oral function evaluation method and so on which can evaluate an oral function of an evaluatee in a simple and easy manner.

### Solution to Problem

An oral function evaluation method according to an aspect of the present invention includes: obtaining voice data obtained by collecting a voice of an evaluatee uttering a syllable or a fixed phrase that includes (i) two or more morae including a change in a first formant frequency or a change in a second formant frequency or (ii) at least one of a flap, a plosive, a voiceless sound, a double consonant, or a fricative; extracting a prosody feature from the voice data obtained; calculating an estimated value of an oral function of the evaluatee, based on the prosody feature extracted and an oral function estimating equation calculated based on a plurality of training data items; and evaluating an oral function deterioration state of the evaluatee by assessing the estimated value using an oral function evaluation indicator.

In addition, a recording medium according to an aspect of the present invention is a non-transitory computer-readable recording medium having recorded thereon a program for causing a computer to execute the oral function evaluation method described above.

In addition, an oral function evaluation device according to an aspect of the present invention includes: an obtainer that obtains voice data obtained by collecting a voice of an evaluatee uttering a syllable or a fixed phrase that includes (i) two or more morae including a change in a first formant frequency or a change in a second formant frequency or (ii) at least one of a flap, a plosive, a voiceless sound, a double consonant, or a fricative; an extractor that extracts a prosody feature from the voice data obtained; a calculator that calculates an estimated value of an oral function of the evaluatee, based on the prosody feature extracted and an oral function estimating equation calculated based on a plurality of training data items; and an evaluator that evaluates an oral function deterioration state of the evaluatee by assessing the estimated value using an oral function evaluation indicator.

In addition, an oral function evaluation system according to an aspect of the present invention includes: the oral function evaluation device described above; and a sound collection device that collects in a contactless manner a voice of the evaluatee uttering the syllable or the fixed phrase.

### Advantageous Effects of Invention

With an oral function evaluation method and so on according to the present invention, it is possible to evaluate an oral function of an evaluatee in a simple and easy manner.

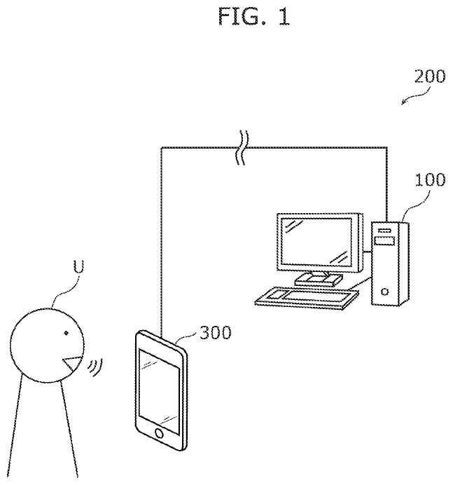

FIG. 1 is a diagram illustrating a configuration of an oral function evaluation system according to an embodiment.

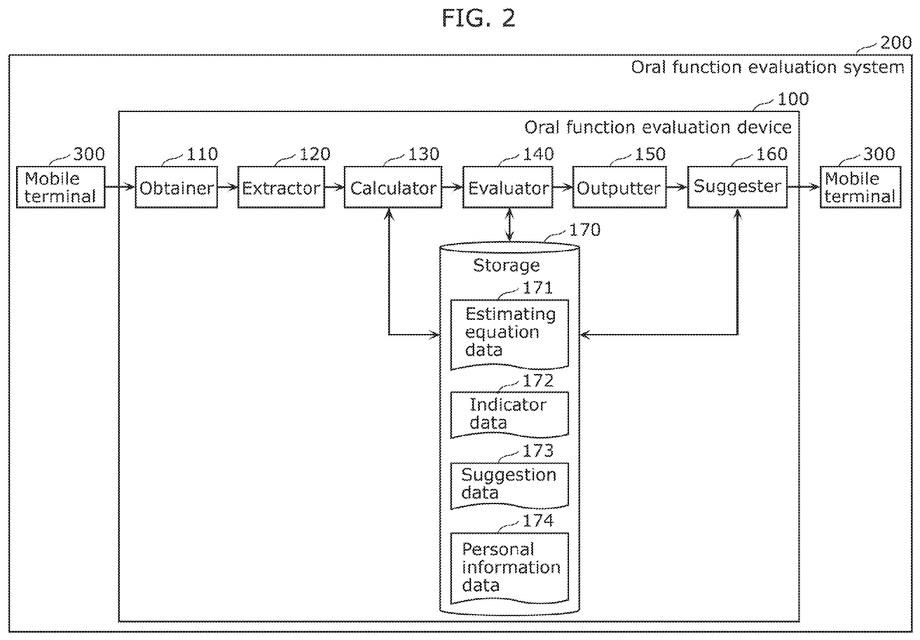

FIG. 2 is a block diagram illustrating a characteristic functional configuration of the oral function evaluation system according to the embodiment.

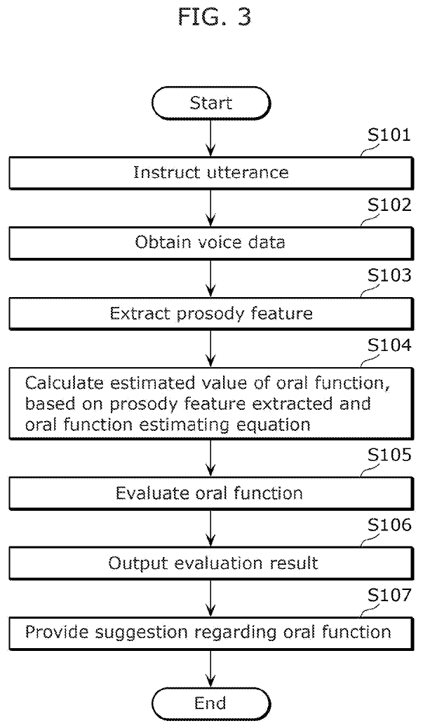

FIG. 3 is a flowchart illustrating a processing procedure for evaluating an oral function of an evaluatee by an oral function evaluation method according to the embodiment.

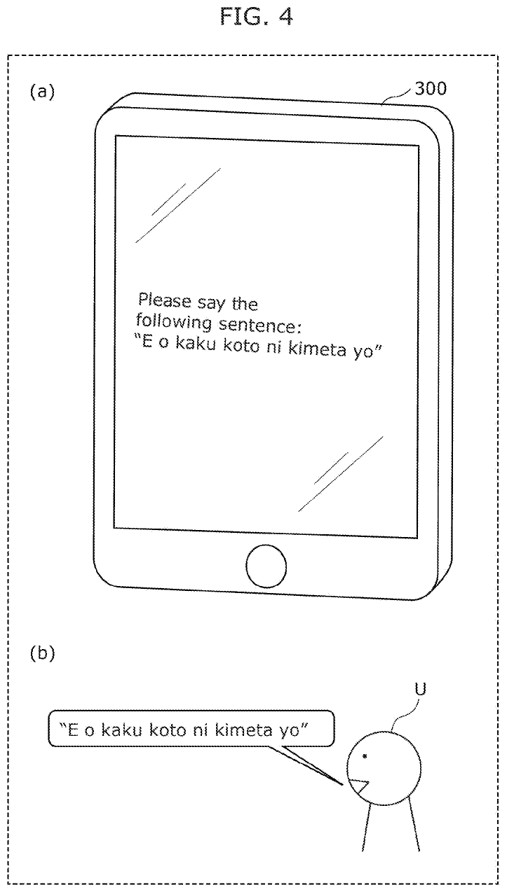

FIG. 4 is a diagram illustrating an outline of a method for obtaining a voice of an evaluatee by the oral function evaluation method according to the embodiment.

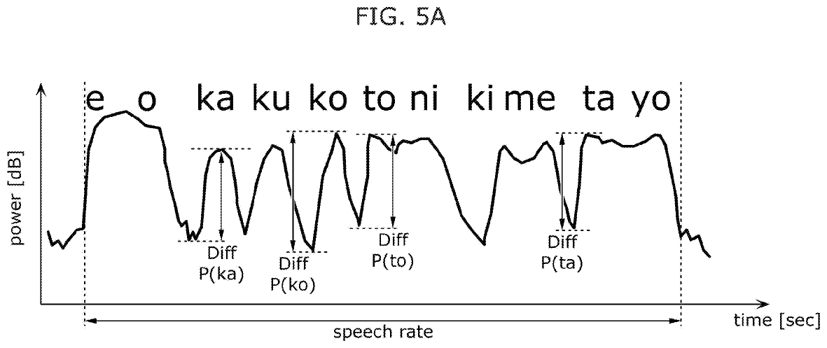

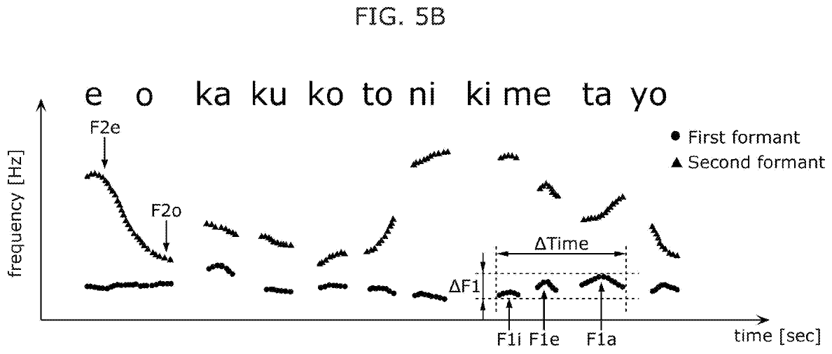

FIG. 5A is a graph illustrating an example of voice data indicating a voice of an evaluatee uttering “e o kaku koto ni kimeta yo.”

FIG. 5B is a graph illustrating an example of changes in formant frequencies of a voice of an evaluatee uttering “e o kaku koto ni kimeta yo.”

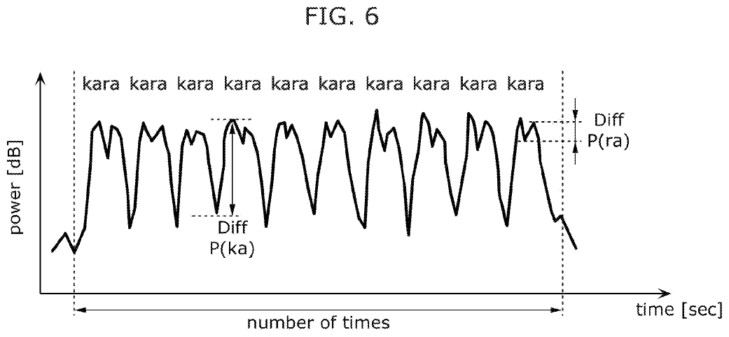

FIG. 6 is a graph illustrating an example of voice data indicating a voice of an evaluatee repeatedly uttering “karakarakara . . . .”

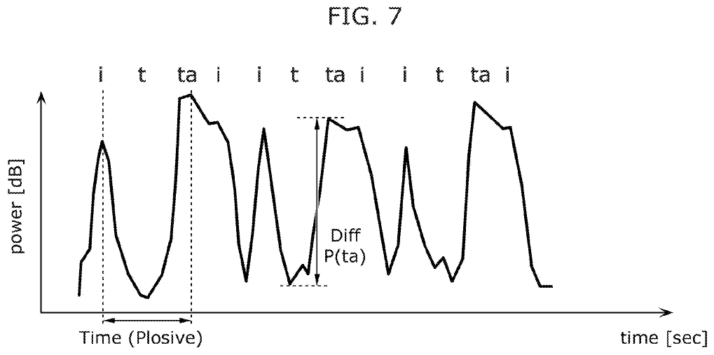

FIG. 7 is a graph illustrating an example of voice data indicating a voice of an evaluatee uttering “ittai.”

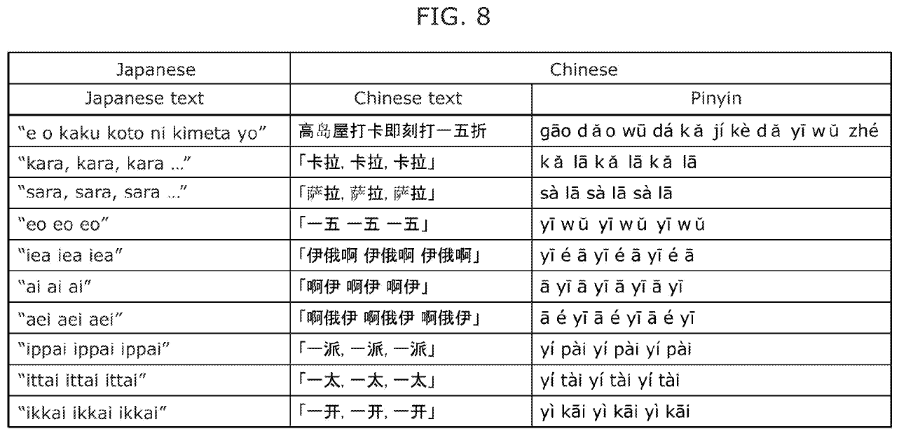

FIG. 8 is a table showing an example of syllables and fixed phrases in Japanese and syllables and fixed phrases in Chinese that are similar in tongue movement or degree of mouth opening and closing when pronounced.

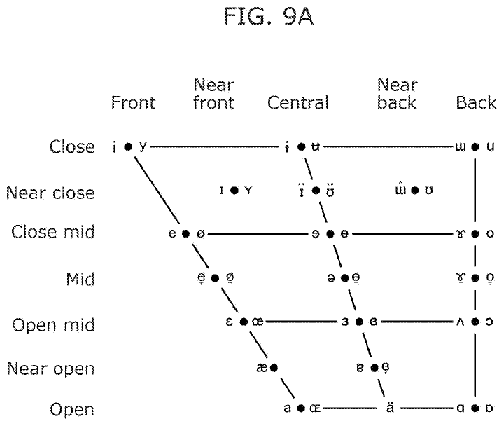

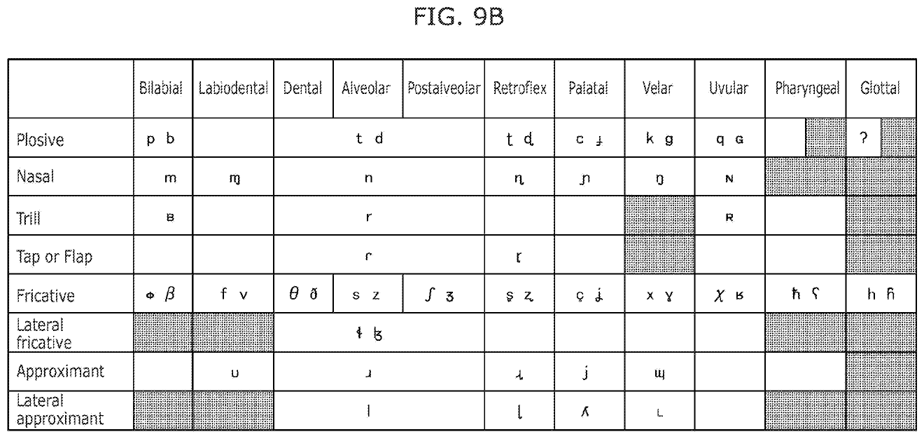

FIG. 9A is a diagram illustrating international phonetic alphabet symbols of vowels.

FIG. 9B is a table illustrating international phonetic alphabet symbols of consonants.

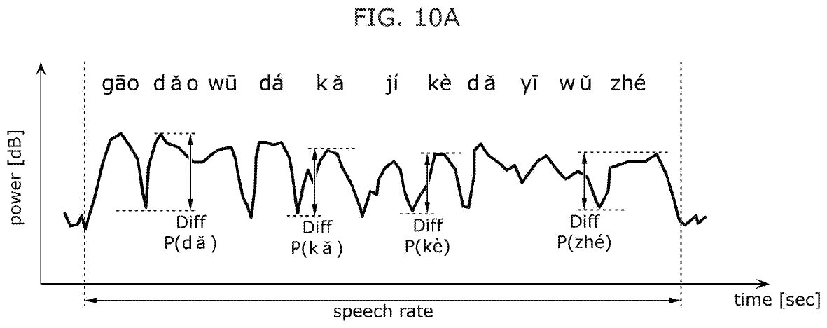

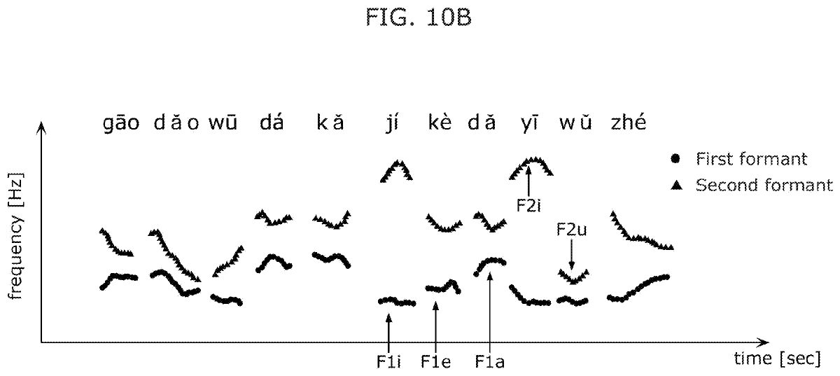

FIG. 10A is a graph illustrating an example of voice data indicating a voice of an evaluatee uttering “gao dao wu da ka ji ke da yi wu zhe.”

FIG. 10B is a graph illustrating an example of changes in formant frequencies of a voice of an evaluatee uttering “gao dao wu da ka ji ke da yi wu zhe.”

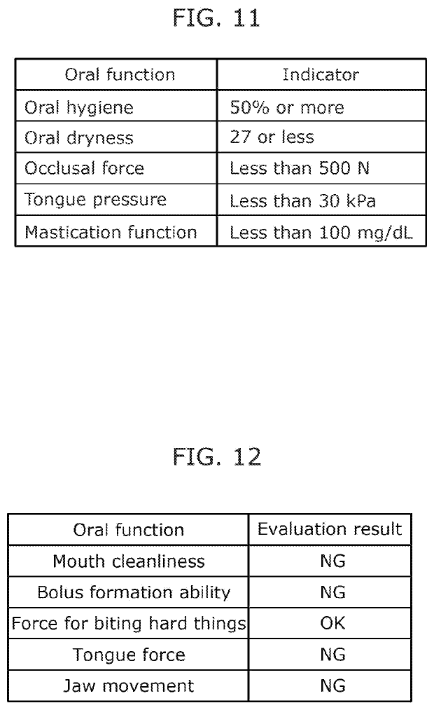

FIG. 11 is a diagram illustrating an example of an oral function evaluation indicator.

FIG. 12 is a table illustrating an example of an evaluation result for elements of an oral function.

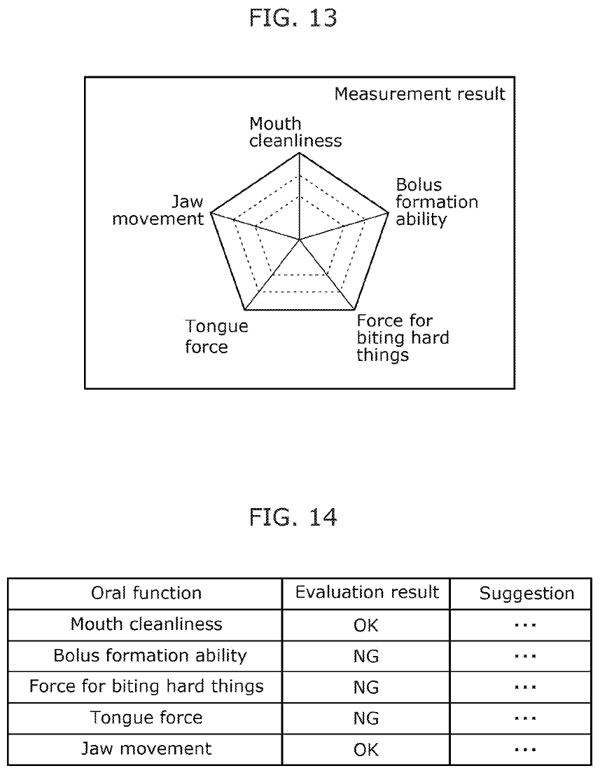

FIG. 13 is a chart illustrating an example of an evaluation result for elements of an oral function.

FIG. 14 is an example of predetermined data that is used when providing a suggestion regarding an oral function.

### DESCRIPTION OF EMBODIMENTS

Hereinafter, embodiments will be described with reference to the drawings. It should be noted that the following embodiments each illustrate a general or specific example. The numerical values, shapes, materials, constituent elements, the arrangement and connection of the constituent elements, steps, the processing order of the steps etc. illustrated in the following embodiments are mere examples, and are not intended to limit the present invention. Among the constituent elements in the following embodiments, those not recited in any of the independent claims representing the most generic concepts will be described as optional constituent elements.

It should be noted that the drawings are represented schematically and are not necessarily precise illustrations. Furthermore, in the drawings, constituent elements that are substantially the same are given the same reference signs, and redundant descriptions will be omitted or simplified.

### Embodiment

### [Elements of Oral Function]

The present disclosure relates to a method for evaluating oral function deterioration and the like, and an oral function includes various elements.

For example, elements of the oral function include tongue fur, oral dryness, occlusal force, tongue pressure, cheek pressure, the remaining number of teeth, swallowing function, mastication function, and so on. The following briefly describes tongue fur, oral dryness, occlusal force, tongue pressure, and mastication function.

The tongue fur indicates how much bacteria or food is deposited on the tongue (i.e., oral hygiene). No tongue fur or thin tongue fur shows that there is an environment of mechanical abrasion (food intake, etc.), cleaning action by saliva is present, or swallowing movement (tongue movement) is normal. In contrast, thick tongue fur shows that poor tongue movement raises a problem in taking food, which may bring about malnutrition or poor muscle strength. The oral dryness is a degree of how dry the tongue is, and when the tongue is dry, movement for speech is inhibited. Food is chewed after being taken into the oral cavity, and the food only chewed is difficult to swallow. Thus, to make it easy to swallow chewed food, saliva exercises a function of gathering the chewed food. However, when the oral cavity is dry, it is difficult to form a bolus (chewed food gathered). The occlusal force is the force for biting hard things and is the strength of jaw muscles. The tongue pressure is an indicator that expresses the force of the tongue pressing the palate. When the tongue pressure is weakened, it may be difficult to make movement of swallowing. Furthermore, when the tongue pressure is weakened, the speed of moving the tongue may decrease, and the speech rate may decrease. The mastication function is a comprehensive function of the oral cavity.

According to the present disclosure, it is possible to evaluate an oral function deterioration state (e.g., a deterioration state of an element of an oral function) of an evaluatee from a voice uttered by the evaluatee. This is because a voice uttered by an evaluatee whose oral function is deteriorating has a specific feature, and by extracting the specific feature as a prosody feature, the oral function of the evaluatee can be evaluated. The present disclosure is implemented by an oral function evaluation method, a program that causes a computer to perform the method, an oral function evaluation device that is an example of the computer, and an oral function evaluation system that includes the oral function evaluation device. Hereinafter, the oral function evaluation method and the like will be described along with the oral function evaluation system.

### [Configuration of Oral Function Evaluation System]

A configuration of oral function evaluation system 200 according to an embodiment will be described.

FIG. 1 is a diagram illustrating a configuration of oral function evaluation system 200 according to the embodiment.

Oral function evaluation system 200 is a system for evaluating an oral function of evaluatee U by analyzing a voice of evaluatee U. As illustrated in FIG. 1, oral function evaluation system 200 includes oral function evaluation device 100 and mobile terminal 300.

Oral function evaluation device 100 is a device that obtains voice data indicating voice uttered by evaluatee U through mobile terminal 300 and evaluates an oral function of evaluatee U from the obtained voice data.

Mobile terminal 300 is a sound collection device that collects in a contactless manner a voice of evaluatee U uttering a syllable or a fixed phrase that includes (i) two or more morae including a change in a first formant frequency or a change in a second formant frequency or (ii) at least one of a flap, a plosive, a voiceless sound, a double consonant, or a fricative, and outputs voice data indicating the collected voice to oral function evaluation device 100. For example, mobile terminal 300 is a smartphone or a tablet computer including a microphone. It should be noted that mobile terminal 300 is not limited to a smartphone, a tablet computer, or the like. Mobile terminal 300 may be, for example, a note PC that is a device having a sound collecting function. Oral function evaluation system 200 may include a sound collecting device (a microphone) instead of mobile terminal 300. Oral function evaluation system 200 may include an input interface for obtaining personal information on evaluatee U. The input interface is not limited to a particular input interface and may be any input interface having an inputting function, such as a keyboard and a touch panel. A volume of the microphone may be set in oral function evaluation system 200.

Mobile terminal 300 may be a display device that includes a display and displays, for example, an image based on image data output from oral function evaluation device 100. It should be noted that the display device need not be mobile terminal 300 and may be a monitor device that includes a liquid crystal panel, an organic EL panel, or the like. In other words, although mobile terminal 300 serves as both a sound collecting device and a display device in the present embodiment, the sound collecting device (microphone), the input interface, and the display device may be provided separately.

It suffices that oral function evaluation device 100 and mobile terminal 300 are capable of transmitting and receiving, for example, voice data or image data for displaying an image indicating an evaluation result described later. Thus, oral function evaluation device 100 and mobile terminal 300 may be connected together in either a wired manner or a wireless manner.

Oral function evaluation device 100 analyzes a voice of evaluatee U based on voice data collected by mobile terminal 300, evaluates an oral function of evaluatee U from a result of the analysis, and outputs an evaluation result. For example, oral function evaluation device 100 outputs, to mobile terminal 300, image data for displaying an image indicating the evaluation result or data for providing a suggestion to evaluatee U that is regarding the oral function and generated based on the evaluation result. With this configuration, oral function evaluation device 100 can notify evaluatee U of a level of an oral function of evaluatee U and a suggestion for preventing the oral function from deteriorating, for example. Thus, for example, evaluatee U can prevent the oral function from deteriorating or improve the oral function.

It should be noted that although oral function evaluation device 100 is, for example, a personal computer, it may be a server device. Further, oral function evaluation device 100 may be mobile terminal 300. That is to say, mobile terminal 300 may have the function of oral function evaluation device 100 described below.

FIG. 2 is a block diagram illustrating a characteristic functional configuration of oral function evaluation system 200 according to the embodiment. Oral function evaluation device 100 includes obtainer 110, extractor 120, calculator 130, evaluator 140, outputter 150, suggester 160, and storage 170.

Obtainer 110 obtains voice data obtained by mobile terminal 300 collecting in a contactless manner a voice uttered by evaluatee U. The voice is a voice of evaluatee U uttering a syllable or a fixed phrase that includes two or more morae including a change in the first formant frequency or a change in the second formant frequency. Alternatively, the voice is a voice of evaluatee U uttering a syllable or a fixed phrase that includes at least one of a flap, a plosive, a voiceless sound, a double consonant, or a fricative. Obtainer 110 may further obtain personal information on evaluatee U. For example, the personal information is information input to mobile terminal 300 and includes an age, weight, height, sex, body mass index (BMI), dental information (e.g., the number of teeth, presence or absence of denture, occlusal support location, the number of functional teeth, the remaining number of teeth, etc.), a value of serum albumin, or an eating rate. The personal information may be obtained through a swallowing screening tool called the eating assessment tool-10 (EAT-10), Seirei dysphagia screening questionnaire, interview, Barthel Index, or the like. In Japan, the personal information may be obtained through Kihon Checklist developed by the Japanese Ministry of Health, Labor and Welfare instead of these. Obtainer 110 is, for example, a communication interface that performs wired communication or wireless communication.

Extractor 120 is a processing unit that analyzes voice data on evaluatee U obtained by obtainer 110. Specifically, extractor 120 is implemented by a processor, a microcomputer, or a dedicated circuit.

Extractor 120 calculates a prosody feature from voice data obtained by obtainer 110. The prosody feature is a numerical value indicating a feature of a voice of evaluatee U that is extracted from voice data used by evaluator 140 to evaluate the oral function of evaluatee U. The prosody feature may include at least one of the speech rate, a sound pressure difference, a change over time in the sound pressure difference, the first formant frequency, the second formant frequency, an amount of change in the first formant frequency, an amount of change in the second formant frequency, a change over time in the first formant frequency, a change over time in the second formant frequency, or a time length of a plosive.

Calculator 130 calculates an estimated value of an oral function of evaluatee U, based on the prosody feature extracted by extractor 120 and an oral function estimating equation calculated based on a plurality of training data items. Estimating equation data 171 indicating the estimating equation is stored in storage 170. Specifically, calculator 130 is implemented by a processor, a microcomputer, or a dedicated circuit.

Evaluator 140 evaluates an oral function deterioration state of evaluatee U by assessing, using an oral function evaluation indicator, the estimated value calculated by calculator 130. Indicator data 172 indicating the oral function evaluation indicator is stored in storage 170. Specifically, evaluator 140 is implemented by a processor, a microcomputer, or a dedicated circuit.

Outputter 150 outputs the estimated value calculated by calculator 130 to suggester 160. Outputter 150 may output an evaluation result on an oral function of evaluatee U evaluated by evaluator 140 to mobile terminal 300, for example. Specifically, outputter 150 is implemented by a processor, a microcomputer, or a dedicated circuit, and a communication interface that performs wired communication or wireless communication.

Suggester 160 provides a suggestion regarding an oral function of evaluatee U by checking the estimated value calculated by calculator 130 against predetermined data. Suggestion data 173, which is the predetermined data, is stored in storage 170. Suggester 160 may provide a suggestion regarding the oral cavity to evaluatee U by checking the personal information obtained by obtainer 110 against suggestion data 173. Suggester 160 outputs the suggestion to mobile terminal 300. Suggester 160 is implemented by, for example, a processor, a microcomputer, or a dedicated circuit, and a communication interface that performs wired communication or wireless communication.

Storage 170 is a storage device in which the following data are stored: estimating equation data 171 indicating the oral function estimating equations calculated based on a plurality of training data items; indicator data 172 indicating the oral function evaluation indicator used for assessing the estimated value of an oral function of evaluatee U; suggestion data 173 indicating a relationship between the estimated value of an oral function and suggestion details; and personal information data 174 indicating the personal information on evaluatee U. Estimating equation data 171 is referred to by calculator 130 when an estimated value of the oral function of evaluatee U is calculated. Indicator data 172 is referred to by evaluator 140 when an oral function deterioration state of evaluatee U is evaluated. Suggestion data 173 is referred to by suggester 160 when a suggestion that is regarding the oral function is provided to evaluatee U. Personal information data 174 is, for example, data obtained via obtainer 110. Personal information data 174 may be stored in storage 170 in advance. Storage 170 is implemented by, for example, read only memory (ROM), random access memory (RAM), semiconductor memory, hard disk drive (HDD), or the like.

Storage 170 may store a program executed by a computer to implement extractor 120, calculator 130, evaluator 140, outputter 150, and suggester 160, image data indicating an evaluation result on the oral function of evaluatee U used when the evaluation result is output, and data including an image, a video, a voice, or a text indicating details of the suggestion. Storage 170 may also store an instruction image described later.

Although not illustrated, oral function evaluation device 100 may include an instructor for instructing evaluatee U to utter a syllable or a fixed phrase that includes (i) two or more morae including a change in the first formant frequency or a change in the second formant frequency or (ii) at least one of a flap, a plosive, a voiceless sound, a double consonant, or a fricative. Specifically, the instructor obtains image data on an instruction image or voice data on an instruction voice that is stored in storage 170 and is for instructing to utter the syllable or the fixed phrase, and outputs the image data or the voice data to mobile terminal 300.

### [Processing Procedure of Oral Function Evaluation Method]

Subsequently, a specific processing procedure in an oral function evaluation method to be executed by oral function evaluation device 100 will be described.

FIG. 3 is a flowchart illustrating a processing procedure for evaluating the oral function of evaluatee U by the oral function evaluation method according to the embodiment. FIG. 4 is a diagram illustrating an outline of a method for obtaining a voice of evaluatee U by the oral function evaluation method.

First, the instructor instructs evaluatee U to utter a syllable or a fixed phrase that includes (i) two or more morae including a change in the first formant frequency or a change in the second formant frequency or (ii) at least one of a flap, a plosive, a voiceless sound, a double consonant, or a fricative (step S101). For example, in step S101, the instructor obtains image data on an instruction image that is for evaluatee U and stored in storage 170, and outputs the image data to mobile terminal 300. Then, as illustrated in (a) of FIG. 4, the instruction image for evaluatee U is displayed on mobile terminal 300. In (a) of FIG. 4, “E o kaku koto ni kimeta yo” is shown as an example of the fixed phrase. However, instructions to utter a fixed phrase such as “Hana saka jiisan to saru kani kassen”, “Hanabi no e o kaku”, or “Himawari ga saita” may be provided. Alternatively, instructions to utter syllables such as “ippai,” “ittai,” “ikkai,” “pattan,” “kappa,” “shippo,” “kikkari,” or “katteni” may be provided. Alternatively, instructions to utter syllables such as “kara,” “sara,” “chara,” “jara,” “shara,” “kyara,” or “pura” may be provided. Alternatively, instructions to utter syllables such as “aei,” “iea,” “ai,” “ia,” “kakeki,” “kikeka,” “naneni,” “chiteta,” “papepi,” “pipepa,” “katepi,” “chipeka,” “kaki,” “tachi,” “papi,” “misa,” “rari,” “wani,” “niwa,” “eo,” “io,” “iu,” “teko,” “kiro,” “teru”, “peko,” “memo,” “emo” may be provided. The instructions to utter may be instructions about repeatedly uttering one of these sets of syllables.

The instructor may also obtain voice data on an instruction voice for evaluatee U stored in storage 170 and output the voice data to mobile terminal 300, thereby providing the instructions described above using the instruction voice for instructing to perform utterance, without using the instruction image for instructing to perform utterance. Alternatively, the instructions may be provided to evaluatee U by an evaluating person (a member of his/her family, a doctor, etc.) who intends to evaluate the oral function of evaluatee U with his/her own voice, without using the instruction image and the instruction voice for instructing to perform utterance.

For example, the syllable or the fixed phrase uttered may include a combination of two or more vowels or a vowel and a consonant. Here, the combination involves mouth opening and closing or back and forth tongue movement for utterance. “E o kaku koto ni kimeta yo” in Japanese is an example of such syllables or a fixed phrase. Uttering “e o” in “e o kaku koto ni kimeta yo” involves back and forth tongue movement, and uttering “kimeta” in “e o kaku koto ni kimeta yo” involves mouth opening and closing. The part “e o” in “e o kaku koto ni kimeta yo” includes second formant frequencies of the vowel “e” and the vowel “o,” and includes an amount of change in the second formant frequency because the vowel “e” and the vowel “o” adjoin each other. This part also includes a change over time in the second formant frequency. The part “kimeta” in “e o kaku koto ni kimeta yo” includes first formant frequencies of the vowel “i,” the vowel “e,” and the vowel “a,” and includes amounts of change in the first formant frequency because the vowel “i,” the vowel “e,” and the vowel “a” adjoin one another. This part also includes changes over time in the first formant frequency. Uttering “e o kaku koto ni kimeta yo” enables extraction of prosody features such as sound pressure differences, the first formant frequencies, the second formant frequencies, the amounts of change in the first formant frequency, the amounts of change in the second formant frequency, the changes over time in the first formant frequency, the changes over time in the second formant frequency, the speech rate, and the like.

For example, the fixed phrase uttered may include repetition of syllables including a flap and a consonant different from the flap. “Karakarakara . . . ” in Japanese is an example of such a fixed phrase. Repeatedly uttering “karakarakara . . . ” enables extraction of a prosody feature such as sound pressure differences, changes over time in the sound pressure difference, changes over time in sound pressure, the number of repetitions, and the like.

For example, the syllable or the fixed phrase uttered may include at least one combination of a vowel and a plosive. “Ittai” in Japanese is an example of such syllables. Uttering “ittai” enables extraction of a prosody feature such as sound pressure differences, a time length of a plosive (a time length between vowels), and the like.

It should be noted that the voice data may be obtained by collecting a voice of evaluatee U uttering the syllable or the fixed phrase at least twice at different speech rates. For example, evaluatee U is instructed to utter “e o kaku koto ni kimeta yo” at a usual speech rate and at a speech rate faster than the usual speech rate. Uttering “e o kaku koto ni kimeta yo” at the usual speech rate and at the speech rate faster than the usual speech rate enables estimation of a degree of keeping a state of the oral function.

Next, as illustrated in FIG. 3, obtainer 110 obtains the voice data on evaluatee U instructed in step S101 via mobile terminal 300 (step S102). As illustrated in (b) of FIG. 4, in step S102, for example, evaluatee U utters syllables or a fixed phrase such as “e o kaku koto ni kimeta yo” toward mobile terminal 300. Obtainer 110 obtains the syllables or the fixed phrase uttered by evaluatee U as the voice data.

Next, extractor 120 extracts a prosody feature from the voice data obtained by obtainer 110 (step S103).

For example, when the voice data obtained by obtainer 110 is voice data obtained from a voice of evaluatee u uttering “e o kaku koto ni kimeta yo,” extractor 120 extracts, as the prosody features, a sound pressure difference, the first formant frequency, the second formant frequency, an amount of change in the first formant frequency, an amount of change in the second formant frequency, a change over time in the first formant frequency, a change over time in the second formant frequency, and the speech rate. This will be described with reference to FIG. 5A and FIG. 5B.

FIG. 5A is a graph illustrating an example of voice data indicating a voice of evaluatee U uttering “e o kaku koto ni kimeta yo.” In the graph illustrated in FIG. 5A, its horizontal axis indicates time, and its vertical axis indicates power (sound pressure). The power indicated on the vertical axis of the graph in FIG. 5A is expressed in decibels (dB).

In the graph illustrated in FIG. 5A, changes in sound pressure corresponding to “e,” “o,” “ka,” “ku,” “ko,” “to,” “ni,” “ki,” “me,” “ta,” “yo” are recognized. In step S102 shown in FIG. 3, obtainer 110 obtains the voice data illustrated in FIG. 5A from evaluatee U. For example, extractor 120 extracts, in step S103 shown in FIG. 3, sound pressures of “k” and “a” in “ka,” sound pressures of “k” and “o” in “ko,” sound pressures of “t” and “o” in “to,” and sound pressures of “t” and “a” in “ta” included in the voice data illustrated in FIG. 5A, with a known method. From the extracted sound pressures of “k” and “a,” extractor 120 extracts sound pressure difference Diff_P(ka) between “k” and “a” as a prosody feature. Likewise, extractor 120 extracts sound pressure difference Diff_P(ko) between “k” and “o,” sound pressure difference Diff_P(to) between “t” and “o,” and sound pressure difference Diff_P(ta) between “t” and “a” as prosody features. For example, with the sound pressure difference, an oral function regarding swallowing force (a pressure of the tongue in contact with the palate) or bolus formation ability can be evaluated. In addition, with the sound pressure difference including “k,” an oral function regarding an ability to prevent food and drink from flowing into the throat can be evaluated.

FIG. 5B is a graph illustrating an example of changes in formant frequencies of a voice of evaluatee U uttering “e o kaku koto ni kimeta yo.” Specifically, FIG. 5B is a graph for describing an example of changes in the first formant frequency and changes in the second formant frequency.

The first formant frequency is a peak frequency of the amplitude of a human voice that appears first from a low-frequency side. The first formant frequency is known for its tendency to reflect a feature regarding mouth opening and closing. The second formant frequency is a peak frequency of the amplitude of a human voice that appears second from a low-frequency side. The second formant frequency is known for its tendency to reflect an influence regarding back and forth tongue movement.

From the voice data indicating the voice uttered by evaluatee U, extractor 120 extracts a first formant frequency and a second formant frequency of each of the vowels, as prosody features. For example, extractor 120 extracts second formant frequency F2e corresponding to the vowel “e” and second formant frequency F2o corresponding to the vowel “o” in “e o”, as the prosody features. In addition, for example, extractor 120 extracts first formant frequency F1i corresponding to the vowel “i,” first formant frequency F1e corresponding to the vowel “e,” and first formant frequency F1a corresponding to the vowel “a” in “kimeta” as the prosody features.

Extractor 120 further extracts amounts of change in the first formant frequency and amounts of change in the second formant frequency of a string of consecutive vowels, as prosody features. For example, extractor 120 extracts an amount of change between second formant frequency F2e and second formant frequency F2o (F2e-F2o) and amounts of change between first formant frequency F1i, first formant frequency F1e, and first formant frequency F1a (F1e-F1i, F1a-F1e, and F1a-F1i), as the prosody features.

Extractor 120 further extracts changes over time in the first formant frequency and changes over time in the second formant frequency of a string of consecutive vowels, as prosody features. For example, extractor 120 extracts a change over time from second formant frequency F2e to second formant frequency F2o and a change over time from first formant frequency F1i through first formant frequency F1e to first formant frequency F1a, as the prosody features. FIG. 5B illustrates an example of the change over time from first formant frequency F1i through first formant frequency F1e to first formant frequency F1a, where the change over time is ΔF1/ΔTime. ΔF1 is F1a-F1i.

For example, with the second formant frequency, an amount of change in the second formant frequency, or a change over time in the second formant frequency, an oral function regarding movement of gathering food (tongue movement in all directions) can be evaluated. In addition, for example, with the first formant frequency, an amount of change in the first formant frequency, or a change over time in the first formant frequency, an oral function regarding an ability to chew food can be evaluated. In addition, with a change over time in the first formant frequency, an oral function regarding an ability to move the mouth quickly can be evaluated.

Extractor 120 may also extract the speech rate as a prosody feature, as illustrated in FIG. 5A. For example, extractor 120 may extract, as the prosody feature, a time length from a start to an end of utterance of “e o kaku koto ni kimeta yo” by evaluatee U. For example, extractor 120 may also extract, as the prosody features, a time length from a start to an end of utterance of a given part of “e o kaku koto ni kimeta yo” rather than the time length to finish utterance of the entire “e o kaku koto ni kimeta yo.” For example, extractor 120 may also extract, as the prosody feature, an average time length taken to utter the entire “e o kaku koto ni kimeta yo” or a word or words in a given part of “e o kaku koto ni kimeta yo.” For example, with the speech rate, an oral function regarding movement of swallowing, movement of gathering food, or tongue dexterity can be evaluated.

For example, when the voice data obtained by obtainer 110 is voice data obtained from a voice of repeatedly uttering “karakarakara . . . ,” extractor 120 extracts changes over time in sound pressure difference as the prosody feature. This will be described with reference to FIG. 6.

FIG. 6 is a graph illustrating an example of voice data indicating a voice of evaluatee U repeatedly uttering “karakarakara . . . .” In the graph illustrated in FIG. 6, its horizontal axis indicates time, and its vertical axis indicates power (sound pressure). The power indicated on the vertical axis of the graph in FIG. 6 is expressed in decibels (dB).

In the graph illustrated in FIG. 6, changes in sound pressure corresponding to “ka” and “ra” are recognized. In step S102 shown in FIG. 3, obtainer 110 obtains the voice data illustrated in FIG. 6 from evaluatee U. For example, extractor 120 extracts, in step S103 shown in FIG. 3, sound pressures of “k” and “a” in “ka” and sound pressures of “r” and “a” in “ra” included in the voice data illustrated in FIG. 6, with a known method. From the extracted sound pressures of “k” and “a,” extractor 120 extracts sound pressure difference Diff_P(ka) between “k” and “a” as a prosody feature. Likewise, extractor 120 extracts sound pressure difference Diff_P(ra) between “r” and “a” as a prosody feature. For example, extractor 120 extracts sound pressure difference Diff_P(ka) and sound pressure difference Diff_P(ra) as the prosody features from each of repeatedly uttered “kara.” Then, extractor 120 extracts a change over time in sound pressure difference Diff_P(ka) as a prosody feature from each of sound pressure differences Diff_P(ka) extracted and extracts a change over time in sound pressure difference Diff_P(ra) as a prosody feature from each of sound pressure differences Diff_P(ra) extracted. For example, with the changes over time in the sound pressure difference, an oral function regarding movement of swallowing, movement of gathering food, or an ability to chew food can be evaluated.

Extractor 120 may extract a change over time in sound pressure as a prosody feature. For example, in each of “kara” in the repeatedly uttered “karakarakara . . . ,” a change over time in minimum sound pressure (sound pressure of “k”) may be extracted, a change over time in maximum sound pressure (sound pressure of “a”) may be extracted, or a change over time in sound pressure between “ka” and “ra” (sound pressure of “r”) may be extracted. For example, with the changes over time in sound pressure, an oral function regarding movement of swallowing, movement of gathering food, or an ability to chew food can be evaluated.

As illustrated in FIG. 6, extractor 120 may also extract the number of repetitions that is the number of times “kara” uttered per given time period as a feature. The given time period is not limited to a particular time period. For example, the given time period is five seconds. For example, with the number of repetitions per given time period, an oral function regarding movement of swallowing or movement of gathering food can be evaluated.

For example, when the voice data obtained by obtainer 110 is voice data obtained from a voice of evaluatee U uttering “ittai,” extractor 120 extracts a sound pressure difference and a time length of a plosive as the prosody features. This will be described with reference to FIG. 7.

FIG. 7 is a graph illustrating an example of voice data indicating a voice of evaluatee U uttering “ittai.” Here, an example of voice data indicating a voice of a repeatedly uttering “ittaiittai” is illustrated. In the graph illustrated in FIG. 7, its horizontal axis indicates time, and its vertical axis indicates power (sound pressure). The power indicated on the vertical axis of the graph in FIG. 7 is expressed in decibels (dB).

In the graph illustrated in FIG. 7, changes in sound pressure corresponding to “i,” “t,” “ta,” and “i” are recognized. In step S102 shown in FIG. 3, obtainer 110 obtains the voice data illustrated in FIG. 7 from evaluatee U. For example, extractor 120 extracts, in step S103 shown in FIG. 3, sound pressures of “t” and “a” in “ta” included in the voice data illustrated in FIG. 7, with a known method. From the extracted sound pressures of “t” and “a,” extractor 120 extracts sound pressure difference Diff_P(ta) between “t” and “a” as a prosody feature. For example, with the sound pressure difference, an oral function regarding swallowing force or bolus formation ability can be evaluated. Extractor 120 also extracts a time length of a plosive Time(i−ta) (a time length of a plosive between “i” and “ta”) as a prosody feature. For example, with the time length of a plosive, an oral function regarding movement of swallowing, movement of gathering food, or stable tongue movement can be evaluated.

The syllables or the fixed phrases uttered described as an example are syllables or fixed phrases in Japanese. However, the syllables or the fixed phrases are not limited to Japanese and may be in any language.

FIG. 8 is a table showing an example of syllables and fixed phrases in Japanese and syllables and fixed phrases in Chinese that are similar in tongue movement or degree of mouth opening and closing when pronounced.

There are various languages in the world, and there are pronunciations that are similar in tongue movement or degree of mouth opening and closing across different languages. For example, a Chinese fixed phrase

[symbol]

[symbol]

(hereafter, will be written as gao dao wu da ka ji ke da yi wu zhe) is similar to a Japanese fixed phrase “e o kaku koto ni kimeta yo” in tongue movement or degree of mouth opening and closing when pronounced and thus enables extraction of prosody features similar to prosody features of the Japanese fixed phrase “e o kaku koto ni kimeta yo.” It should be noted that addition of tonal markers is omitted in the present specification. FIG. 8 shows some examples of pairs of syllables or fixed phrases in Japanese and Chinese that are similar in tongue movement or degree of mouth opening and closing when pronounced, for reference.

The fact that there are pronunciations that are similar in tongue movement or degree of mouth opening and closing across different languages in various languages present in the world will be simply described with reference to FIG. 9A and FIG. 9B.

FIG. 9A is a diagram illustrating international phonetic alphabet symbols of vowels.

FIG. 9B is a table illustrating international phonetic alphabet symbols of consonants.

In a position relationship of the international phonetic alphabet symbols of vowels illustrated in FIG. 9A, the horizontal direction indicates back and forth tongue movement where symbols close to each other are similar in back and forth tongue movement, and the vertical direction indicates a degree of mouth opening and closing where symbols close to each other are similar in degree of mouth opening and closing. In the table of international phonetic alphabet symbols of consonants illustrated in FIG. 9B, the horizontal direction indicates parts from the lips to the throat used in pronunciation, and an international phonetic alphabet symbol present in a cell in the table indicates that the same sound can be pronounced using the same part. For this reason, the present invention is applicable to various languages existing in the world.

For example, when a large mouth opening and closing is intended, syllables or a fixed phrase is to include consecutive international phonetic alphabet symbols that are away from each other in the vertical direction illustrated in FIG. 9A (e.g., “i” and “a”). Accordingly, an amount of change in the first formant frequency as a prosody feature can be increased. For example, when a large back and forth tongue movement is intended, syllables or a fixed phrase is to include consecutive international phonetic alphabet symbols that are away from each other in the horizontal direction illustrated in FIG. 9A (e.g., “i” and “u”). Accordingly, an amount of change in the second formant frequency as a prosody feature can be increased.

For example, when the voice data obtained by obtainer 110 is voice data obtained from a voice of uttering “gao dao wu da ka ji ke da yi wu zhe,” extractor 120 extracts, as prosody features, a sound pressure difference, the first formant frequency, the second formant frequency, an amount of change in the first formant frequency, an amount of change in the second formant frequency, a change over time in the first formant frequency, a change over time in the second formant frequency, and the speech rate. This will be described with reference to FIG. 10A and FIG. 10B.

FIG. 10A is a graph illustrating an example of voice data indicating a voice of evaluatee U uttering “gao dao wu da ka ji ke da yi wu zhe.” In the graph illustrated in FIG. 10A, its horizontal axis indicates time, and its vertical axis indicates power (sound pressure). The power indicated on the vertical axis of the graph in FIG. 10A is expressed in decibels (dB).

In the graph illustrated in FIG. 10A, changes in sound pressure corresponding to “gao,” “dao,” “wu,” “da,” “ka,” “ji,” “ke,” “da,” “yi,” “wu,” and “zhe” are recognized. In step S102 shown in FIG. 3, obtainer 110 obtains the voice data illustrated in FIG. 10A from evaluatee U. For example, extractor 120 extracts, in step S103 shown in FIG. 3, sound pressures of “d” and “a” in “dao,” sound pressures of “k” and “a” in “ka,” sound pressures of “k” and “e” in “ke,” and sound pressures of “zh” and “e” in “zhe” included in the voice data illustrated in FIG. 10A, with a known method. From the extracted sound pressures of “d” and “a,” extractor 120 extracts sound pressure difference Diff_P(da) between “d” and “a” as a prosody feature. Likewise, extractor 120 extracts, as prosody features, sound pressure difference Diff_P(ka) between “k” and “a,” sound pressure difference Diff_P(ke) between “k” and “e,” and sound pressure difference Diff_P(zhe) between “zh” and “e.” For example, with the sound pressure difference, an oral function regarding swallowing force or bolus formation ability can be evaluated. In addition, with the sound pressure difference including “k,” an oral function regarding an ability to prevent food and drink from flowing into the throat can be evaluated.

FIG. 10B is a graph illustrating an example of changes in formant frequencies of a voice of evaluatee U uttering “gao dao wu da ka ji ke da yi wu zhe.” Specifically, FIG. 10B is a graph for describing an example of changes in the first formant frequency and changes in the second formant frequency.

From the voice data indicating the voice uttered by evaluatee U, extractor 120 extracts a first formant frequency and a second formant frequency of each of the vowels, as prosody features. For example, extractor 120 extracts first formant frequency F1i corresponding to the vowel “i” in “ji,” first formant frequency F1e corresponding to the vowel “e” in “ke,” and first formant frequency F1a corresponding to the vowel “a” in “da,” as the prosody features. In addition, for example, extractor 120 extracts second formant frequency F2i corresponding to the vowel “i” in “yi,” and second formant frequency F2u corresponding to the vowel “u” in “wu,” as the prosody features.

Extractor 120 further extracts amounts of change in the first formant frequency and amounts of change in the second formant frequency of a string of consecutive vowels, as prosody features. For example, extractor 120 extracts amounts of change between first formant frequency F1i, first formant frequency F1e, and first formant frequency F1a (F1e-F1i, F1a-F1e, and F1a-F1i) and an amount of change between second formant frequency F2i and second formant frequency F2u (F2i-F2u), as the prosody features.

Extractor 120 further extracts changes over time in the first formant frequency and changes over time in the second formant frequency of a string of consecutive vowels, as prosody features. For example, extractor 120 extracts a change over time from first formant frequency F1i through first formant frequency F1e to first formant frequency F1a and a change over time from second formant frequency F2i to second formant frequency F2u, as the prosody features.

For example, with the second formant frequency, an amount of change in the second formant frequency, or a change over time in the second formant frequency, an oral function regarding movement of gathering food can be evaluated. In addition, for example, with the first formant frequency, an amount of change in the first formant frequency, or a change over time in the first formant frequency, an oral function regarding an ability to chew food can be evaluated. In addition, with a change over time in the first formant frequency, an oral function regarding an ability to move the mouth quickly can be evaluated.

Extractor 120 may also extract the speech rate as a prosody feature, as illustrated in FIG. 10A. For example, extractor 120 may extract, as the prosody feature, a time length from a start to an end of utterance of “gao dao wu da ka ji ke da yi wu zhe” by evaluatee U. For example, extractor 120 may also extract, as the prosody features, a time length from a start to an end of utterance of a given part of “gao dao wu da ka ji ke da yi wu zhe” rather than the time length taken to finish utterance of the entire “gao dao wu da ka ji ke da yi wu zhe.” For example, extractor 120 may also extract, as the prosody feature, an average time length taken to utter the entire “gao dao wu da ka ji ke da yi wu zhe” or a word or words in a given part of “gao dao wu da ka ji ke da yi wu zhe.” For example, with the speech rate, an oral function regarding movement of swallowing, movement of gathering food, or tongue dexterity can be evaluated.

Returning to the description of FIG. 3, calculator 130 calculates an estimated value of an oral function of evaluatee U, based on the prosody feature extracted and an oral function estimating equation calculated based on a plurality of training data items (step S104).

The oral function estimating equation is set in advance based on results of evaluation performed on subjects. Through a statistical analysis of voice features collected from utterances of the subjects and results of actual diagnoses on oral functions of the subjects, the estimating equation is set in the form of a multiple regression equation or the like about correlations between the voice features and the results of the diagnoses. Selection of a voice feature used as a representative value can generate different variations of estimating equations. The estimating equation can be generated in advance in this manner.

Alternatively, the estimating equation may be set using machine learning to express correlations between the voice features and the results of the diagnoses. Techniques of the machine learning include logistic regression, support vector machine (SVM), and random forest.

For example, the estimating equation can include a coefficient corresponding to an element of an oral function and a variable that is substituted by a prosody feature extracted and is multiplied by the coefficient. Equations 1 through 5 shown below are examples of the estimating equations.

$$Estimated value of oral hygiene = ( A 1 \times F 2 e ) + ( B 1 \times F 2 o ) + ( C 1 \times F 1 i ) + ( D 1 \times F 1 e ) + ( E 1 \times F 1 a ) + ( F 1 \times Diff\_P ( ka ) ) + ( G 1 \times Diff\_P ( ko ) ) + ( H1 \times Diff\_P ( to ) ) + ( J 1 \times Diff\_P ( ta ) ) + ( K 1 \times Diff\_P ( ka ) ) + ( L1 \times Diff\_P ( ra ) ) + ( M 1 \times Diff\_P ( ta ) ) + ( N 1 \times Time ( i - ta ) ) + P 1 \qquad ( Equation 1 )$$

$$Estimated value of oral dryness = ( A 2 \times F 2 e ) + ( B 2 \times F 2 o ) + ( C 2 \times F 1 i ) + ( D 2 \times F 1 e ) + ( E 2 \times F 1 a ) + ( F 2 \times Diff\_P ( ka ) ) + ( G 2 \times Diff\_P ( ko ) ) + ( H 2 \times Diff\_P ( to ) ) + ( J2 \times Diff\_P ( ta ) ) + ( K 2 \times Diff\_P ( ka ) ) + ( L 2 \times Diff\_P ( ra ) ) + ( M 2 \times Diff\_P ( ta ) ) + ( N 2 \times Time ( i - ta ) ) + P 2 \qquad ( Equation 2 )$$

$$Estimated value of occlusal force = ( A 3 \times F 2 e ) + ( B 3 \times F 2 o ) + ( C 3 \times F 1 i ) + ( D 3 \times F 1 e ) + ( E3 \times F 1 a ) + ( F 3 \times Diff\_P ( ka ) ) + ( G 3 \times Diff\_P ( ko ) ) + ( H3 \times Diff\_P ( to ) ) + ( J 3 \times Diff\_P ( ta ) ) + ( K 3 \times Diff\_P ( ka ) ) + ( L 3 \times Diff\_P ( ra ) ) + ( M 3 \times Diff\_P ( ta ) ) + ( N 3 \times Time ( i - ta ) ) + P 3 \qquad ( Equation 3 )$$

$$Estimated value of tongue pressure = ( A 4 \times F 2 e ) + ( B 4 \times F 2 o ) + ( C 4 \times F 1 i ) + ( D 4 \times F 1 e ) + ( E 4 \times F 1 a ) + ( F 4 \times Diff\_P ( ka ) ) + ( G 4 \times Diff\_P ( ko ) ) + ( H4 \times Diff\_P ( to ) ) + ( J4 \times Diff\_P ( ta ) ) + ( K4 \times Diff\_P ( ka ) ) + ( L 4 \times Diff\_P ( ra ) ) + ( M4 \times Diff\_P ( ta ) ) + ( N 4 \times Time ( i - ta ) ) + P 4 \qquad ( Equation 4 )$$

$$Estimated value of mastication function = ( A 5 \times F 2 e ) + ( B5 \times F 2 o ) + ( C5 \times F 1 i ) + ( D5 \times F 1 e ) + ( E5 \times F 1 a ) + ( F5 \times Diff\_P ( ka ) ) + ( G5 \times Diff\_P ( ko ) ) + ( H5 \times Diff\_P ( to ) ) + ( J5 \times Diff\_P ( ta ) ) + ( K5 \times Diff\_P ( ka ) ) + ( L5 \times Diff\_P ( ra ) ) + ( M5 \times Diff\_P ( ta ) ) + ( N5 \times Time ( i - ta ) ) + P5 \qquad ( Equation 5 )$$

A1, B1, C1, . . . , N1, A2, B2, C2, . . . , N2, A3, B3, C3, . . . , N3, A4, B4, C4, . . . , N4, A5, B5, C5, . . . , N5 are coefficients, and are specifically coefficients corresponding to elements of the oral function. For example, A1, B1, C1, . . . , N1 are coefficients corresponding to oral hygiene which is one of the elements of the oral function; A2, B2, C2, . . . , N2 are coefficients corresponding to oral dryness which is one of the elements of the oral function; A3, B3, C3, . . . , N3 are coefficients corresponding to occlusal force which is one of the elements of the oral function; A4, B4, C4, . . . , N4 are coefficients corresponding to tongue pressure which is one of the elements of the oral function; and A5, B5, C5, . . . , N5 are coefficients corresponding to mastication function which is one of the elements of the oral function.

P1 is a constant corresponding to oral hygiene, P2 is a constant corresponding to oral dryness, P3 is a constant corresponding to occlusal force, P4 is a constant corresponding to tongue pressure, and P5 is a constant corresponding to mastication function.

F2e multiplied by A1, A2, A3, A4, or A5 and F2o multiplied by B1, B2, B3, B4, or B5 are variables to be substituted by second formant frequencies that are prosody features extracted from utterance data on the utterance of “e o kaku koto ni kimeta yo” by evaluatee U. F1i multiplied by C1, C2, C3, C4, or C5, F1e multiplied by D1, D2, D3, D4, or D5, and F1a multiplied by E1, E2, E3, E4, or E5 are variables to be substituted by first formant frequencies that are prosody features extracted from utterance data on the utterance of “e o kaku koto ni kimeta yo” by evaluatee U. Diff_P(ka) multiplied by F1, F2, F3, F4, or F5, Diff_P(ko) multiplied by G1, G2, G3, G4, or G5, Diff_P(to) multiplied by H1, H2, H3, H4, or H5, and Diff_P(ta) multiplied by J1, J2, J3, J4, or J5 are variables to be substituted by sound pressure differences that are prosody features extracted from utterance data on the utterance of “e o kaku koto ni kimeta yo” by evaluatee U. Diff_P(ka) multiplied by K1, K2, K3, K4, or K5 and Diff_P(ra) multiplied by L1, L2, L3, L4, or L5 are variables to be substituted by sound pressure differences that are prosody features extracted from utterance data on the utterance of “kara” by evaluatee U. Diff_P(ta) multiplied by M1, M2, M3, M4, or M5 is a variable to be substituted by a sound pressure difference that is a prosody feature extracted from utterance data on the utterance of “ittai” by evaluatee U. Time(i−ta) multiplied by N1, N2, N3, N4, or N5 is a variable to be substituted by a time length of a plosive that is a prosody feature extracted from utterance data on the utterance of “ittai” by evaluatee U.

As shown in Equations 1 through 5 above, calculator 130, for example, calculates an estimated value for each of elements (e.g., tongue fur, oral dryness, occlusal force, tongue pressure, and mastication function) of the oral function of evaluatee U. It should be noted that these elements of the oral function are mere examples, and it suffices if the elements of the oral function include at least one of tongue fur, oral dryness, occlusal force, tongue pressure, cheek pressure, the remaining number of teeth, swallowing function, or mastication function of evaluatee U.

In addition, for example, extractor 120 extracts a plurality of prosody features from voice data items obtained by collecting a voice of evaluatee U uttering two or more types of syllables or two or more types of fixed phrases (e.g., “e o kaku koto ni kimeta yo,” “kara,” and “ittai” in Equation 1 through Equation 5 shown above), and calculator 130 calculates an estimated value of an oral function based on the plurality of prosody features extracted and one of the estimating equations. By substituting the plurality of prosody features extracted from the voice data on the two or more types of syllables or two or more types of fixed phrases into one of the estimating equations, calculator 130 can calculate the estimated value of the oral function with high precision.

Although the linear expressions are shown as the estimating equations, the estimating equations may be multidimensional equations such as two-dimensional equations.

Next, evaluator 140 evaluates an oral function deterioration state of evaluatee U by assessing, using an oral function evaluation indicator, the estimated value calculated by calculator 130 (step S105). For example, evaluator 140 evaluates an oral function deterioration state of evaluatee U for each of the elements of the oral function by assessing, using an oral function evaluation indicator determined for each of the elements of the oral function, the estimated value calculated for each of the elements of the oral function. The oral function evaluation indicator is an indicator for evaluating the oral function. For example, the oral function evaluation indicator is a condition for determining that the oral function has deteriorated. The oral function evaluation indicator will be described with reference to FIG. 11.

FIG. 11 is a diagram illustrating an example of the oral function evaluation indicator.

The oral function evaluation indicator is determined for each of the elements of the oral function. For example, in Japan, an indicator of 50% or more is determined for oral hygiene, an indicator of 27 or less is determined for oral dryness, an indicator of less than 500 N is determined for occlusal force (when DENTALPRESCALE II from GC Corporation is used), an indicator of less than 30 kPa is determined for tongue pressure, and an indicator of less than 100 mg/dL is determined for mastication function (for the indicators, see “Koukukinouteikashou ni kansuru kihonteki na kangaekata (in Japanese) (Basic approaches to oral hypofunction) (https://www.jads.jp/basic/pdf/document_02.pdf, https://www.jads.jp/basic/pdf/document-220331-2.pdf)” in Japanese Association for Dental Science). Additionally, for reference, an English paper “Oral hypofunction in the older population: Position paper of the Japanese Society of Gerodontology in 2016 (https://onlinelibrary.wiley.com/doi/epdf/10.1111/ger.12347)” also describes the indicators. It should be noted that the indicator for occlusal force depends on the measurement method, and in the above English paper, the estimated value of the occlusal force is less than 200 N. In the present working example, 500 N, which is mentioned in the Japanese document, is used as an example. Evaluator 140 evaluates an oral function deterioration state of evaluatee U for each of the elements of the oral function by comparing the estimated value calculated for each of the elements of the oral function with the oral function evaluation indicator determined for each of the elements of the oral function. For example, when the estimated value of the oral hygiene calculated is 50% or more, the oral hygiene as an element of the oral function is evaluated as being in a deteriorated state. Likewise, when the estimated value of the oral dryness calculated is 27 or less, the oral dryness as an element of the oral function is evaluated as being in a deteriorated state, when the estimated value of the occlusal force calculated is less than 500 N, the occlusal force as an element of the oral function is evaluated as being in a deteriorated state, when the estimated value of the tongue pressure calculated is less than 30 kPa, the tongue pressure as an element of the oral function is evaluated as being in a deteriorated state, and when the estimated value of the mastication function calculated is less than 100 mg/dL, the mastication function as an element of the oral function is evaluated as being in a deteriorated state. It should be noted that the oral function evaluation indicators shown in FIG. 11 determined for the oral hygiene, the oral dryness, the occlusal force, the tongue pressure, and the mastication function are an example, and the oral function evaluation indicators are not limited to these. For example, an indicator for remaining teeth may be determined for the mastication function. Furthermore, the oral hygiene, the oral dryness, the occlusal force, the tongue pressure, and the mastication function are shown as the elements of the oral function but are an example. For example, for tongue-lip motor hypofunction, there are elements of the oral function including tongue movement, lip movement, and lip strength. It should be noted that the oral function evaluation indicators may differ country by country. Therefore, an oral function evaluation indicator defined by the country where oral function evaluation device 100 is used may be used.

Returning to the description of FIG. 3, outputter 150 outputs an evaluation result on the oral function of evaluatee U evaluated by evaluator 140 (step S106). For example, outputter 150 outputs the evaluation result to mobile terminal 300. In this case, for example, outputter 150 may include a communication interface that performs wired communication or wireless communication. Outputter 150 obtains image data on an image corresponding to the evaluation result from storage 170 and sends the obtained image data to mobile terminal 300. An example of the image data (the evaluation result) is illustrated in FIG. 12 and FIG. 13.

FIG. 12 and FIG. 13 are a table and a chart illustrating the example of the evaluation result for the elements of the oral function. As shown in FIG. 12, the evaluation result may be an evaluation result in two levels: OK and NG. OK means being normal, and NG means being abnormal. Normal or abnormal need not be indicated for each of the elements of the oral function. For example, only an evaluation result of an element that is suspected of deteriorating may be indicated. Furthermore, the evaluation result is not limited to an evaluation result in two levels and may be an evaluation result with three or more fractionalized levels of evaluation. In this case, indicator data 172 stored in storage 170 may include a plurality of indicators for one element. Alternatively, as illustrated in FIG. 13, the evaluation result may be expressed in a radar chart. FIG. 12 and FIG. 13 indicate, as elements of the oral function, mouth cleanliness, bolus formation ability, force for biting hard things, tongue force, and jaw movement. The evaluation result is presented based on the estimated value of the oral hygiene for the mouth cleanliness, the estimated value of the oral dryness for the bolus formation ability, the estimated value of the occlusal force for the force for biting hard things, the estimated value of the tongue pressure for the tongue force, and the estimated value of the mastication function for the jaw movement. It should be noted that FIG. 12 and FIG. 13 are an example, and that wording which describes the evaluation items, items of the oral function, and combinations of such corresponding wording and items are not limited to those in FIG. 12 and FIG. 13.

Returning to the description of FIG. 3, suggester 160 provides a suggestion regarding the oral function of evaluatee U by checking the estimated value calculated by calculator 130 against predetermined data (suggestion data 173) (step S107). Here, the predetermined data will be described with reference to FIG. 14.

FIG. 14 is an example of predetermined data (suggestion data 173) that is used when providing a suggestion regarding the oral function.

As illustrated in FIG. 14, suggestion data 173 is data in which an evaluation result and details of a suggestion are associated with each other for each of the elements of the oral function. For example, when the estimated value of the mouth cleanliness calculated is 50% or more, the indicator is satisfied. Therefore, suggester 160 determines the mouth cleanliness to be OK and provides a suggestion based on details of the suggestion associated with the mouth cleanliness. Descriptions of specific details of suggestions are omitted. For example, storage 170 includes data indicating the details of suggestions (e.g., images, videos, voices, texts, etc.), and suggester 160 provides a suggestion regarding the oral function to evaluatee U using such data.

### Advantageous Effects Etc.

As described above, the oral function evaluation method according to the present embodiment includes: obtaining voice data obtained by collecting a voice of an evaluatee uttering a syllable or a fixed phrase that includes (i) two or more morae including a change in a first formant frequency or a change in a second formant frequency or (ii) at least one of a flap, a plosive, a voiceless sound, a double consonant, or a fricative (step S102); extracting a prosody feature from the voice data obtained (step S103); calculating an estimated value of an oral function of the evaluatee, based on the prosody feature extracted and an oral function estimating equation calculated based on a plurality of training data items (step S104); and evaluating an oral function deterioration state of the evaluatee by assessing the estimated value using an oral function evaluation indicator (step S105).

Accordingly, obtaining voice data suitable for the evaluation of the oral function enables easy evaluation of the oral function of evaluatee U. In other words, simply by evaluatee U uttering the syllables or the fixed phrase toward the sound collecting device of mobile terminal 300 or the like, the oral function of evaluatee U can be evaluated. In particular, since the estimated values of the oral function are calculated using the estimating equations that are calculated based on a plurality of training data items, an oral function deterioration state can be evaluated quantitatively. Furthermore, the estimated values are calculated from the prosody features and the estimating equations, and the estimated values are compared with threshold values (the oral function evaluation indicators) rather than comparing the prosody features directly with the threshold values to evaluate the oral function. Therefore, the oral function deterioration state can be evaluated with high precision.

For example, the oral function estimating equation may include a coefficient corresponding to an element of an oral function and a variable that is substituted by the prosody feature extracted and is multiplied by the coefficient.

Accordingly, the estimated values of the oral function can be easily calculated simply by substituting the extracted prosody features into the estimating equations.

For example, in the calculating, the estimated value may be calculated for each of elements of the oral function of evaluatee U, and in the evaluating, an oral function deterioration state of evaluatee U may be evaluated for each of the elements of the oral function by assessing, using an oral function evaluation indicator determined for each of the elements of the oral function, the estimated value calculated for each of the elements of the oral function.

Accordingly, the oral function deterioration state can be evaluated for each element. For example, by preparing estimating equations that include coefficients that differ among the elements of the oral function for the elements of the oral function, the oral function deterioration state can be easily evaluated for each element.

For example, the elements of the oral function may include at least one of tongue fur, oral dryness, occlusal force, tongue pressure, cheek pressure, a remaining number of teeth, swallowing function, or mastication function of evaluatee U.

Accordingly, it is possible to evaluate a deterioration state regarding at least one element of the oral function among tongue fur, oral dryness, occlusal force, tongue pressure, cheek pressure, a remaining number of teeth, swallowing function, or mastication function of evaluatee U.

For example, the prosody feature may include at least one of a speech rate, a sound pressure difference, a change over time in the sound pressure difference, the first formant frequency, the second formant frequency, an amount of change in the first formant frequency, an amount of change in the second formant frequency, a change over time in the first formant frequency, a change over time in the second formant frequency, or a time length of a plosive.

Oral function deterioration causes a change in pronunciation. Therefore, the oral function deterioration state can be evaluated from these prosody features.

For example, in the extracting, a plurality of prosody features may be extracted from the voice data obtained by collecting a voice of evaluatee U uttering two or more types of the syllable or two or more types of the fixed phrase, and in the calculating, the estimated value may be calculated based on the plurality of prosody features extracted and the oral function estimating equation.

Accordingly, by using the plurality of prosody features extracted for one estimating equation based on the two or more types of syllables or two or more types of fixed phrases, precision of the calculation of the estimated value of the oral function can be increased.

For example, the syllable or the fixed phrase may include a combination of two or more vowels or a vowel and a consonant. Here, the combination involves mouth opening and closing or back and forth tongue movement for utterance.

Accordingly, a prosody feature including an amount of change in the first formant frequency, a change over time in the first formant frequency, an amount of change in the second formant frequency, or a change over time in the second formant frequency can be extracted from the voice of evaluatee U uttering such a syllable or fixed phrase.

For example, the voice data may be obtained by collecting a voice of evaluatee U uttering the syllable or the fixed phrase at least twice at different speech rates.

Accordingly, a degree of keeping a state of the oral function can be estimated from a voice of evaluatee U uttering such syllables or fixed phrases.

For example, the fixed phrase may include repetition of syllables including a flap and a consonant different from the flap.

Accordingly, prosody features including a change over time in sound pressure difference, a change over time in sound pressure, and the number of repetitions can be extracted from the voice of evaluatee U uttering such syllables or a fixed phrase.

For example, the syllable or the fixed phrase may include at least one combination of a vowel and a plosive.

Accordingly, prosody features including a sound pressure difference and a time length of a plosive can be extracted from a voice of evaluatee U uttering such a syllable or fixed phrase.

For example, the oral function evaluation method may further include providing a suggestion regarding the oral function of evaluatee U by checking the estimated value against predetermined data.

Accordingly, evaluatee U can be provided with a suggestion about how to take measures against oral function deterioration.

Oral function evaluation device 100 according to the present embodiment includes: obtainer 110 that obtains voice data obtained by collecting a voice of evaluatee U uttering a syllable or a fixed phrase that includes (i) two or more morae including a change in a first formant frequency or a change in a second formant frequency or (ii) at least one of a flap, a plosive, a voiceless sound, a double consonant, or a fricative; extractor 120 that extracts a prosody feature from the voice data obtained; calculator 130 that calculates an estimated value of an oral function of evaluatee U, based on the prosody feature extracted and an oral function estimating equation calculated based on a plurality of training data items; and evaluator 140 that evaluates an oral function deterioration state of evaluatee U by assessing the estimated value using an oral function evaluation indicator.

Accordingly, it is possible to provide oral function evaluation device 100 capable of evaluating an oral function of evaluatee U in a simple and easy manner.

Oral function evaluation system 200 according to the present embodiment includes oral function evaluation device 100 and a sound collection device (mobile terminal 300) that collects in a contactless manner a voice of evaluatee U uttering a syllable or a fixed phrase.

Accordingly, it is possible to provide oral function evaluation system 200 capable of evaluating an oral function of evaluatee U in a simple and easy manner.

### Other Embodiments

The oral function evaluation method and so on according to the present embodiment have been described above, but the present invention is not limited to the above embodiment.

For example, estimating equation data 171 may be updated based on an evaluation result obtained by a specialist actually diagnosing the oral function of evaluatee U. Accordingly, precision of the evaluation of the oral function can be increased. Machine learning may be used to increase the precision of the evaluation of the oral function.

For example, the details of suggestion may be evaluated by evaluatee U, and suggestion data 173 may be updated based on an evaluation result. For example, in a case where a suggestion about an oral function that is unproblematic for evaluatee U, evaluatee U evaluates that details of the suggestion are wrong. Then, by updating suggestion data 173 based on the evaluation result, a wrong suggestion as described above is inhibited from being provided. In this manner, details of a suggestion regarding an oral function for evaluatee U can be made more effective. Machine learning may be used to make details of a suggestion regarding an oral function more effective.

For example, evaluation results on oral functions may be accumulated together with personal information items as big data, and the big data may be used for the machine learning. Furthermore, details of suggestions regarding oral functions may be accumulated together with personal information items as big data, and the big data may be used for the machine learning.

Further, for example, although the oral function evaluation method in the above embodiment includes providing a suggestion regarding an oral function (step S107), this process need not be included. In other words, oral function evaluation device 100 need not include suggester 160.

Further, for example, although the personal information on evaluatee U is obtained in obtaining voice data (step S102) in the above embodiment, the personal information on evaluatee U need not be obtained. In other words, obtainer 110 need not obtain the personal information on evaluatee U.

Further, for example, the steps included in the oral function evaluation method may be executed by a computer (a computer system). The present invention can be implemented as a program for causing a computer to execute the steps included in the oral function evaluation method. In addition, the present invention can be implemented as a non-transitory computer-readable recording medium such as a CD-ROM having such a program recorded thereon.

For example, in the case where the present invention is implemented using a program (a software product), each step is performed as a result of the program being executed using hardware resources such as a CPU, memory, and an input and output circuit of a computer. That is to say, each step is performed by the CPU obtaining data from, for example, the memory or the input and output circuit and performing calculation on the data, and outputting the calculation result to the memory or the input and output circuit, for example.

Further, each of the constituent elements included in oral function evaluation device 100 and oral function evaluation system 200 according to the above embodiment may be implemented as a dedicated or general-purpose circuit.

Further, each of the constituent elements included in oral function evaluation device 100 and oral function evaluation system 200 according to the above embodiment may be implemented as a large-scale integrated (LSI) circuit, which is an integrated circuit (IC).

Such IC is not limited to an LSI, and thus may be implemented as a dedicated circuit or a general-purpose processor. A field programmable gate array (FPGA) that allows for programming, or a reconfigurable processor that allows for reconfiguration of the connection and the setting of circuit cells inside an LSI may be employed.

Furthermore, when advancement in semiconductor technology or derivatives of other technologies brings forth a circuit integration technology which replaces LSI, it will be appreciated that such a circuit integration technology may be used to integrate the constituent elements included in oral function evaluation device 100 and oral function evaluation system 200.

The present invention also includes other forms achieved by making various modifications to the embodiments that may be conceived by those skilled in the art, as well as forms implemented by arbitrarily combining the constituent elements and functions in each embodiment without materially departing from the essence of the present invention.

1. An oral function evaluation method comprising:
obtaining voice data obtained by collecting a voice of an evaluatee uttering a syllable or a fixed phrase that includes (i) two or more morae including a change in a first formant frequency or a change in a second formant frequency or (ii) at least one of a flap, a plosive, a voiceless sound, a double consonant, or a fricative;
extracting a prosody feature from the voice data obtained;
calculating an estimated value of an oral function of the evaluatee, based on the prosody feature extracted and an oral function estimating equation calculated based on a plurality of training data items; and
evaluating an oral function deterioration state of the evaluatee by assessing the estimated value using an oral function evaluation indicator.

2. The oral function evaluation method according to claim 1,
wherein the oral function estimating equation includes a coefficient corresponding to an element of an oral function and a variable that is substituted by the prosody feature extracted and is multiplied by the coefficient.

3. The oral function evaluation method according to claim 1,
wherein in the calculating, the estimated value is calculated for each of elements of the oral function of the evaluatee, and
in the evaluating, an oral function deterioration state of the evaluatee is evaluated for each of the elements of the oral function by assessing, using an oral function evaluation indicator determined for each of the elements of the oral function, the estimated value calculated for each of the elements of the oral function.

4. The oral function evaluation method according to claim 1,
wherein elements of the oral function include at least one of tongue fur, oral dryness, occlusal force, tongue pressure, cheek pressure, a remaining number of teeth, swallowing function, or mastication function of the evaluatee.

5. The oral function evaluation method according to claim 1,
wherein the prosody feature includes at least one of a speech rate, a sound pressure difference, a change over time in the sound pressure difference, the first formant frequency, the second formant frequency, an amount of change in the first formant frequency, an amount of change in the second formant frequency, a change over time in the first formant frequency, a change over time in the second formant frequency, or a time length of a plosive.

6. The oral function evaluation method according to claim 1,
wherein in the extracting, a plurality of prosody features are extracted from the voice data obtained by collecting a voice of the evaluatee uttering two or more types of the syllable or two or more types of the fixed phrase, and
in the calculating, the estimated value is calculated based on the plurality of prosody features extracted and the oral function estimating equation.

7. The oral function evaluation method according to claim 1,
wherein the syllable or the fixed phrase includes a combination of two or more vowels or a vowel and a consonant, the combination involving mouth opening and closing or back and forth tongue movement for utterance.

8. The oral function evaluation method according to claim 1,
wherein the voice data is obtained by collecting a voice of the evaluatee uttering the syllable or the fixed phrase at least twice at different speech rates.

9. The oral function evaluation method according to claim 1,
wherein the fixed phrase includes repetition of syllables including a flap and a consonant different from the flap.

10. The oral function evaluation method according to claim 1,
wherein the syllable or the fixed phrase includes at least one combination of a vowel and a plosive.

11. The oral function evaluation method according to claim 1, further comprising:
providing a suggestion regarding the oral function of the evaluatee by checking the estimated value against predetermined data.

12. A non-transitory computer-readable recording medium having recorded thereon a program for causing a computer to execute the oral function evaluation method according to claim 1.

13. An oral function evaluation device comprising:
an obtainer that obtains voice data obtained by collecting a voice of an evaluatee uttering a syllable or a fixed phrase that includes (i) two or more morae including a change in a first formant frequency or a change in a second formant frequency or (ii) at least one of a flap, a plosive, a voiceless sound, a double consonant, or a fricative;
an extractor that extracts a prosody feature from the voice data obtained;
a calculator that calculates an estimated value of an oral function of the evaluatee, based on the prosody feature extracted and an oral function estimating equation calculated based on a plurality of training data items; and
an evaluator that evaluates an oral function deterioration state of the evaluatee by assessing the estimated value using an oral function evaluation indicator.

14. An oral function evaluation system comprising:
the oral function evaluation device according to claim 13; and
a sound collection device that collects in a contactless manner a voice of the evaluatee uttering the syllable or the fixed phrase.
